(19) United States
(12) Patent Application Publication
(10) Pub. No.: US 2017/0049482 A1
(43) Pub. Date: Feb. 23, 2017

(54) SPINAL SCREWS AND METHODS OF USING THE SAME

(71) Applicant: Spinal USA, Inc., Parsippany, NJ (US)

(72) Inventors: Michael N. Campbell, Florham Park, NJ (US); Christopher DeNicola, Mt. Arlington, NJ (US)

(21) Appl. No.: 15/239,627

(22) Filed: Aug. 17, 2016

Related U.S. Application Data
(60) Provisional application No. 62/206,179, filed on Aug. 17, 2015.
(60) Provisional application No. 62/217,673, filed on Sep. 11, 2015.

(51) Int. Cl. A61B 17/70; A61B 17/86

(52) U.S. Cl. CPC A61B 17/7032; A61B 17/866

(57) ABSTRACT

Bone screw assemblies that can include a screw, housing, snap ring, saddle, set screw, and rod are described herein. The bone screw assemblies can allow for polyaxial or uniplanr movement of the screw relative to the housing. A threaded portion of the screw can be coated with a hydroxyapatite (HA) coating. The screw can have a triple lead thread configuration. The bone screw assembly may be modular.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. The present application claims priority to U.S. Provisional Application Nos. 62/206,179, filed Aug. 17, 2015, and 62/217,673, filed Sep. 11, 2015, the entirety of each of which is hereby incorporated by reference herein.

### BACKGROUND

Field

The present application generally relates to devices and methods for treating the spine. In particular, certain embodiments of the present application relate to spinal screws, such as pedicle screws, and methods of use.

Description of the Related Art

Spinal fusion encompasses a surgical technique in which two or more vertebrae are connected together. This technique may be used for multiple indications, including abnormal spinal curvature (e.g., scoliosis) and weakening or injuring of the vertebrae or spinal disc.

In some instances, this process is accomplished and/or supplemented using a plurality of screws implanted into the pedicles of adjacent vertebrae and joined together by a series of one or more rods. The pedicle screw may have an enlarged head that interfaces with a housing having a corresponding cavity, thus allowing for a range of polyaxial or uniplanar articulation between the screw and the housing. After the pedicle screw is implanted into bone, a rod may be placed in the housing, and a set screw may be delivered into engagement with the housing, applying a downward force on the rod to hold the assembly together.

### SUMMARY

In some embodiments, a pedicle screw includes a screw and a housing. The screw has a threaded shaft coated with a hydroxyapatite coating and an enlarged head at a proximal end. The housing has an upper portion with an upper opening and a lower portion with a lower opening extending along a first axis of the housing. The enlarged head of the screw is disposed within the housing and the shaft extends out of the housing through the lower opening. The housing also has a third opening and a fourth opening along a second axis transverse to the first axis adapted to receive an elongated rod.

In some embodiments, the threaded shaft of the screw has a triple lead thread configuration. The pedicle screw can include a snap ring positioned around the enlarged head of the screw and in the lower portion of the housing adjacent the lower opening. The pedicle screw can include a saddle disposed in the housing that has a generally cylindrical outer surface, an upper surface having a partially cylindrically shaped indentation configured to receive the elongated rod, and a lower surface having an indentation configured to receive the enlarged head. In some embodiments, the upper portion of the housing is internally threaded and configured to receive an externally threaded set screw. In some embodiments, the screw can rotate and pivot polyaxially relative to the housing prior to the rod being secured within the housing. Alternatively, in some embodiments, the screw can rotate and pivot uniaxially relative to the housing prior to the rod being secured within the housing. In some embodiments, the housing includes two extended tabs extending from opposite sides of the housing that are configured to guide the rod into the housing. The extended tabs can be internally threaded.

In some embodiments, only a portion of the shaft of the screw is coated with the hydroxyapatite coating. In some embodiments, a distal portion of the shaft has a relatively thinner hydroxyapatite coating, and a proximal portion of the shaft has a relatively thicker hydroxyapatite coating. In some embodiments, surface of each thread revolution of the shaft is uniformly coated with the hydroxyapatite coating. In other embodiments, a thickness of the hydroxyapatite coating on proximally-facing flanks is different than a thickness of the hydroxyapatite coating on distally-facing flanks. In some embodiments, only a crest and either a proximally-facing flank or a distally-facing flank of each thread revolution of the shaft is coated with the hydroxyapatite coating.

In some embodiments, a method of manufacturing a bone screw assembly includes providing a bone screw and a housing, wherein the bone screw comprises an enlarged head and a shaft having a threaded portion, and wherein the housing is configured to receive the bone screw such that the enlarged head is disposed in a lower portion of the housing and the shaft extends from a lower opening of the housing, and applying a hydroxyapatite coating to the threaded portion of the bone screw. In some embodiments, applying the hydroxyapatite coating includes spray coating the threaded portion of the bone screw with the hydroxyapatite coating.

In some embodiments, a modular pedicle screw assembly incudes a screw and a housing provided separately from the screw. The screw has a threaded shaft and an enlarged head at a proximal end. The housing has an upper portion with an upper opening and a lower portion with a lower opening extending along a first axis of the housing; the housing also has a third opening and a fourth opening along a second axis transverse to the first axis adapted to receive an elongated rod. The housing is configured to be secured to the screw when the screw is secured to a patient's vertebra such that the enlarged head of the screw is disposed within the housing and the shaft extends out of the housing through the lower opening. In some embodiments, the modular pedicle screw assembly further includes a snap ring provided with the housing and positioned in the lower portion of the housing. The snap ring is configured to be positioned around the enlarged head of the screw when the housing is secured to the screw. An interior surface of the lower portion of the housing can be tapered. An outer surface of the snap ring can be tapered.

In some embodiments, a method of securing a pedicle screw to a patient's vertebra includes securing a screw to the vertebra and securing a housing to the screw when the screw is secured to the vertebra. The screw has a threaded shaft and an enlarged head at a proximal end. The housing is provided separately from the housing and has an upper portion with an upper opening and a lower portion with a lower opening extending along a first axis of the housing, and a third opening and a fourth opening along a second axis transverse to the first axis adapted to receive an elongated rod. When the housing is secured to the screw, the enlarged head of the screw is disposed within the housing and the shaft extends out of the housing through the lower opening. In some embodiments, a snap ring is provided with the housing and positioned in the lower portion of the housing. The snap ring is configured to be positioned around the enlarged head of the screw when the housing is secured to the screw.

All of these embodiments are intended to be within the scope of the disclosure herein. These and other embodiments will become readily apparent to those skilled in the art from the following detailed description having reference to the attached figures, the disclosure not being limited to any particular disclosed embodiment(s).

### BRIEF DESCRIPTION OF THE DRAWINGS

These and other features, aspects, and advantages of the present disclosure are described with reference to the drawings of certain embodiments, which are intended to schematically illustrate certain embodiments and not to limit the disclosure.

FIG. 1 shows an exploded view of an example embodiment of a screw assembly including a screw, housing, snap ring, and saddle;

FIG. 2A shows an assembled cross-sectional view of the screw assembly of FIG. 1;

FIG. 2B shows a detail assembled cross-sectional view of a portion of the screw assembly of FIGS. 1 and 2A;

FIG. 3 shows a cross-sectional view of a distal portion of the screw of the screw assembly of FIGS. 1-2B;

FIG. 4A shows a perspective view of the housing of the screw assembly of FIGS. 1-2B;

FIG. 4B shows a cross-sectional view of the housing of FIG. 4A;

FIG. 5A shows a perspective view of the snap ring of the screw assembly of FIGS. 1-2B;

FIG. 5B shows a top view of the snap ring of FIG. 5A;

FIG. 5C shows a cross-sectional view of the snap ring of FIGS. 5A-5B;

FIG. 6 shows an exploded view of another example embodiment of a screw assembly including a screw, housing, snap ring, and saddle;

FIG. 7A shows an assembled cross-sectional view of the screw assembly of FIG. 6;

FIG. 7B shows a detail assembled cross-sectional view of a portion of the screw assembly of FIGS. 6 and 7A;

FIG. 8A shows the screw of the screw assembly of FIGS. 6-7B;

FIG. 8B shows a detail view of a head portion of the screw of FIG. 8A;

FIG. 9A shows a perspective view of the housing of the screw assembly of FIGS. 6-7B;

FIG. 9B shows a cross-sectional view of the housing of FIGS. 9A;

FIG. 10A shows a top view of the snap ring of the screw assembly of FIGS. 6-7B;

FIG. 10B shows a perspective view of the snap ring of FIG. 10A;

FIG. 11A shows a perspective view of an example embodiment of a housing for a reduction screw assembly;

FIG. 11B shows a perspective view of another example embodiment of a housing for a reduction screw assembly;

FIG. 11C shows a cross-sectional view of the housing of FIG. 11B;

FIG. 11D shows a detail cross-sectional view of the housing of FIGS. 11B-11C;

FIG. 12 shows a cross-sectional view of an example embodiment of a housing for a modular screw assembly;

FIG. 13 shows a cross-sectional view of an example embodiment of a housing for a modular reduction screw assembly;

FIG. 14A shows a side view of an example embodiment of a snap ring for a modular screw assembly;

FIG. 14B shows a top view of the snap ring of FIG. 14A; and

FIG. 14C shows a cross-sectional view of the snap ring of FIGS. 14A-14B.

### DETAILED DESCRIPTION

Although certain embodiments and examples are described below, those of skill in the art will appreciate that the disclosure extends beyond the specifically disclosed embodiments and/or uses and obvious modifications and equivalents thereof Thus, it is intended that the scope of the disclosure herein disclosed should not be limited by any particular embodiments described below.

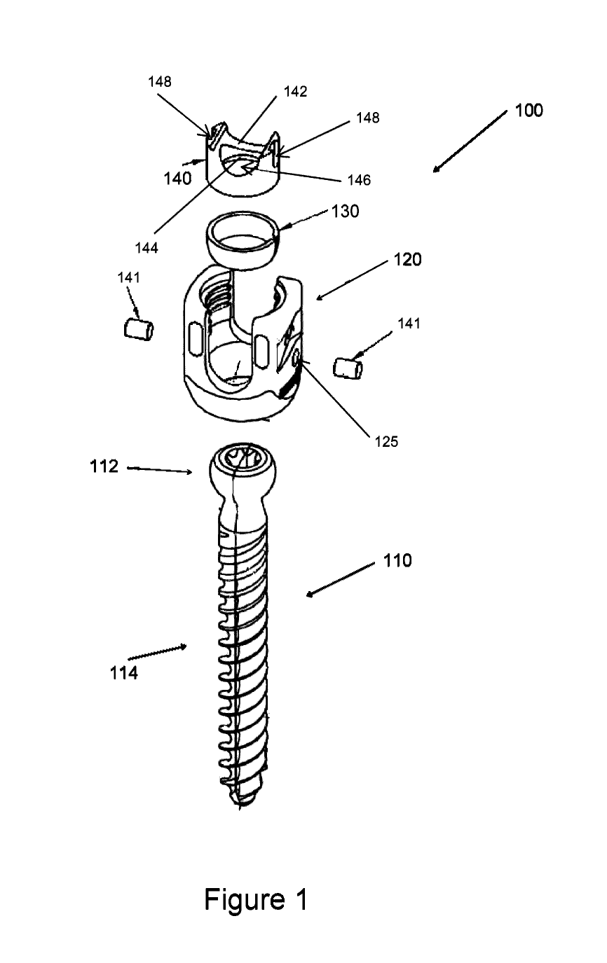

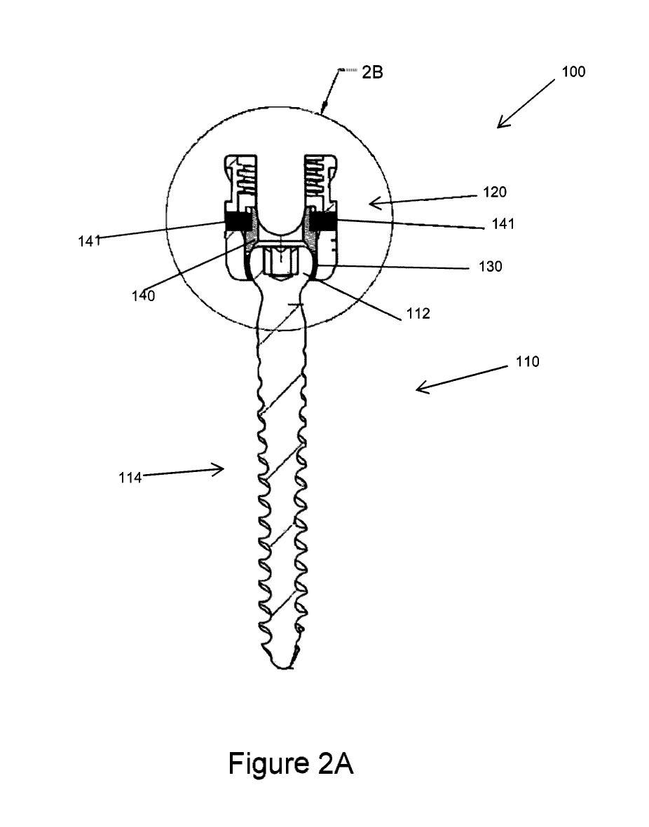

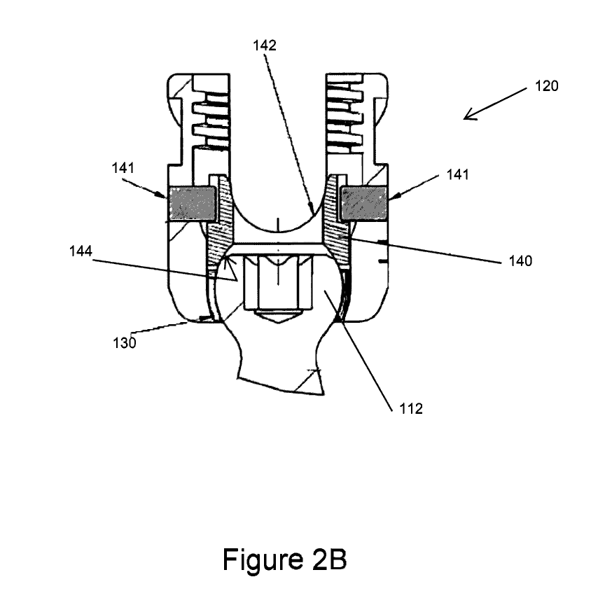

As shown in FIGS. 1-2B, an example embodiment of a screw assembly 100 can include a screw 110 configured to be secured to a vertebra, a housing 120, a snap ring 130, and a saddle 140. The screw assembly 100 can further include a set screw and a spinal rod. In some embodiments, one or more of the components described herein is made of a metal, such as titanium or alloys thereof For example, one or more components can be made at least partially of titanium 6AL 4V ELI. In some embodiments, the housing 120 is made of cobalt-chrome. In some embodiments, the spinal rod can be made of cobalt-chrome and/or titanium or a titanium alloy.

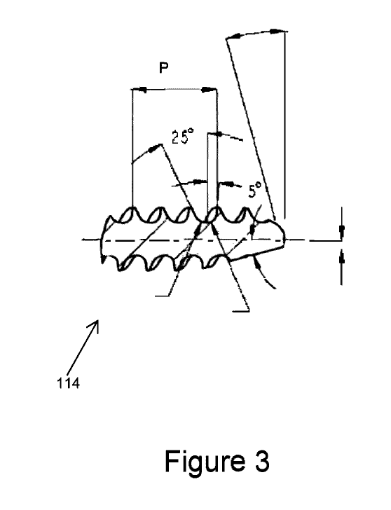

The screw 110 has an enlarged head 112 at a proximal end and a shaft or body portion 114 extending from the head 112 to a tip at a distal end. The head 112 can be approximately spherical or ball-shaped. In the illustrated embodiment, the head 112 is partially spherical and has a flattened proximal end or surface that can receive a screwdriver. The shaft 114 can be at least partially threaded and adapted to be implanted into a patient's spine, for example, into the pedicle of a vertebra. In the illustrated embodiment, the screw 110 is self-tapping and is not cannulated. However, in other embodiments, the screw 110 may be non-self-tapping and/or cannulated. In some embodiments, for example as shown in FIGS. 1-3, the shaft 114 has a triple lead thread configuration. In other words, the shaft 114 has three thread leads or starts proximate the distal end. The three leads can be spaced apart by about 120°, although other spacing is also possible. The overall pitch P, indicated in FIG. 3, of the threaded portion of the shaft can be in the range of about 0.284 in. to about 0.294 in. The triple lead configuration provides greater axial movement of the screw 110 per rotation, so the screw 110 can advantageously be inserted into the bone more quickly and easily. In the illustrated embodiment, the threads are proximally tapered such that the threads become shallower toward the proximal end of the shaft 114. This advantageously increases the interface between the bone and screw 110 and enhances pull-out strength.

At least a portion of the screw shaft 114 can be coated with a hydroxyapatite (HA) coating. The HA coating can advantageously promote bone growth and bonding between the shaft 114 and the bone. The HA coating can advantageously enhance the amount of fixation possible between the screw 110 and the surrounding bone, particularly, for example, in osteoporotic patients. In some embodiments, the entirety of the threaded portion of the shaft 114 can be coated with the HA coating, with the enlarged head and the unthreaded neck between the enlarged head and the threaded portion being uncoated. In some embodiments, the HA coating does not extend proximally more than about 0.080 in. from a proximalmost thread or end of the threaded portion. Otherwise, the HA coating may interfere with rotation of the screw 110 relative to the housing 120 as described in greater detail herein. During application of the HA coating, the screw 110, particularly the enlarged head 112 and any portion of the shaft 114 more than about 0.080 in. proximal of the proximalmost thread or end of the threaded portion can be masked to prevent or inhibit the coating from applied to those portions of the screw 110. In other embodiments, not all of the threaded portion of the shaft 114 is coated. For example, the distal tip of the shaft 114 (e.g., 1, 2 or 3 turns of the threaded portion) may be uncoated.

The HA coating can be applied to a thickness in the range of about 40 μm to about 60 μm. The HA coating may be uniform or variable over the portion of the shaft being coated. In some embodiments, the HA coating can be thinner at or near the distal tip (e.g., below about 40 μm), such that there is a relatively thinner HA coating along a distal portion of the shaft, and a relatively thicker HA coating along a proximal portion of the shaft. In some embodiments, the distalmost 1, 2 or 3 turns of the threads of the shaft 114 may have a thinner HA coating.

The HA coating may be applied uniformly or non-uniformly along the surface of each thread revolution, such that in some embodiments, the crest, root and flanks of each thread have the same or approximately the same thickness HA coating, and in other embodiments, the thicknesses of HA coating may vary (for example, by having relatively larger or smaller thickness HA coating on a proximally-facing flank versus the distally facing flank). In even further embodiments, some but not all of the surfaces of the thread have an HA coating (e.g., just the crest and one of the flanks).

In some embodiments, the shaft 114 is spray coated with the HA coating. In some such embodiments, the HA coating is primarily sprayed onto the shaft 114 proximal to the distal tip and the distal tip receives overspray of the HA coating. In other embodiments, the HA coating may be applied by dip coating or other processes. When applied, the HA coating should be free of scratches, voids, and chips, and should be able to withstand gamma sterilization (e.g., at an 80 kGy maximum dose) exposure without mechanical non-conformities. The HA coating is in compliance with ASTM F1185-03 and ISO 13779-2 for chemical analysis, elements, crystalline phase composition, crystallinity value, and adhesion to substrate.

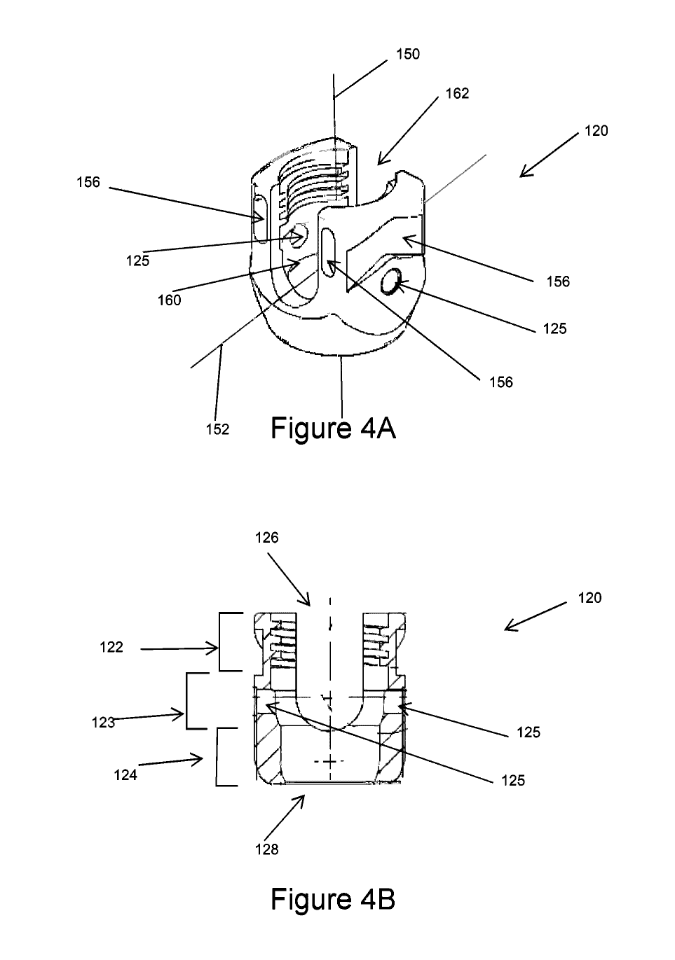

As shown in FIGS. 4A-4B, the housing 120 includes an upper portion 122 having an upper opening 126, a lower portion 124 having a lower opening 128, and an intermediate portion 123. The upper opening 126 and lower opening 128 can extend along a first axis 150 of the housing 120. The upper opening 126 and lower opening 128 can be connected so as to create a through hole passing from the upper opening 126, through the upper portion 126, intermediate portion 123, and lower portion 124, to the lower opening 128. In use, the screw 110 is disposed within the housing 120 such that the head 112 is within the lower portion 124 and the shaft 114 extends through the lower opening 128, as shown in FIGS. 2A-2B. A diameter of the upper opening 126 can be greater than a diameter of the lower opening 128. A diameter of the enlarged head 112 of the screw 110 can be smaller than the diameter of the upper opening 126 and the diameter of the lower opening 128. The screw 110 can therefore be loaded into the housing 120 from either the upper opening 126 or the lower opening 128. In the illustrated embodiment, an interior of the upper portion 122 is generally cylindrical. An interior surface of the lower portion 124, or at least a lower section of the lower portion 124, can have a gradually decreasing diameter towards the bottom of the housing 120. The interior surface of the lower portion 124 or lower section of the lower portion 124 can be conical, tapered, or curved. In the illustrated embodiment, an interior surface of an upper section of the lower portion 124 is generally cylindrical, and the interior surface of the lower section of the lower portion 124 is curved.

The housing 120 further includes a third opening 160 and a fourth opening 162 extending along a second axis 152 of the housing 120 that is transverse to the first axis 150. The third opening 160 and fourth opening 162 intersect an upper edge of the housing 120 and separate the upper portion 122 and intermediate portion 123 of the housing 120 into two opposing arms. In the illustrated embodiment, the third opening 160 and fourth opening 162 are generally U-shaped, although other shapes are also possible. In use, the third opening 160 and fourth opening 162 receive the rod such that the rod is disposed within the intermediate portion 123, and lower or distal portions of the third opening 160 and fourth opening 162 define a seat for the rod.

In the illustrated embodiment, the upper portion 122 of the housing 120 is internally threaded to receive and engage an externally threaded set screw. The threading may not extend below a point at or below the rod when the rod is disposed in the housing 120 in use. In other embodiments, the upper portion 122 may be externally threaded to receive and engage an internally threaded set screw or cap, or the upper portion 122 may receive and engage a closure mechanism via means other than threading. The set screw can have square or modified square threads, although other types of threads are also possible. The intermediate portion 123 can include one or more holes 125 extending perpendicularly to the first axis 150 and second axis 152. In the illustrated embodiment, the intermediate portion 123 includes two holes 125 positioned opposite each other with one hole 125 through each of the arms of the housing 120. An outer surface of the housing 120 can include one or more indentations 156 that receive an insertion tool during use.

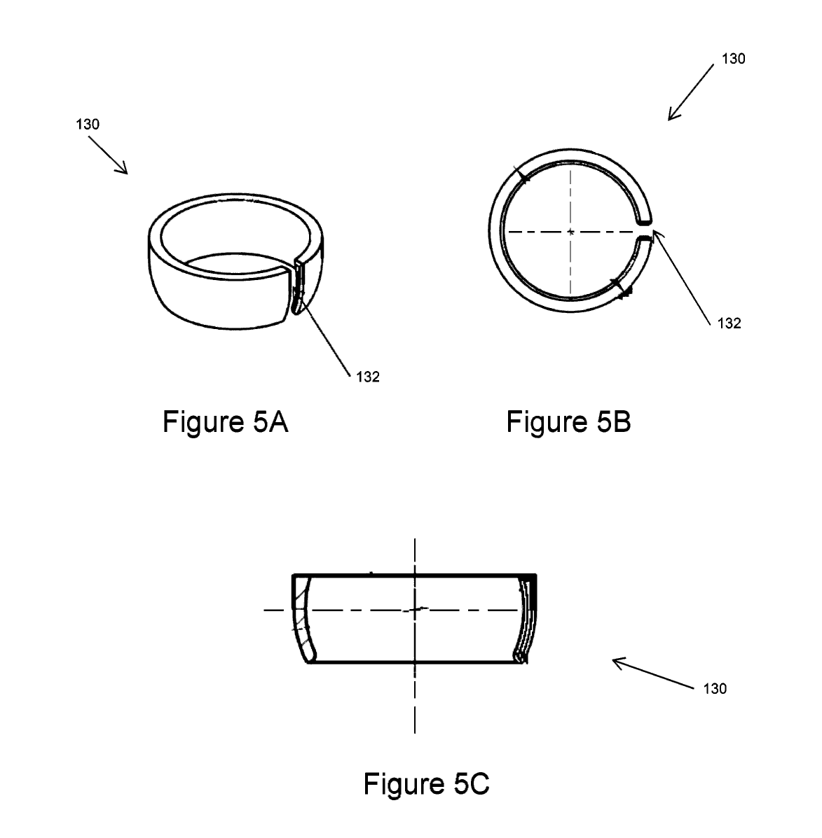

As shown in FIGS. 5A-5C, the snap ring 130 is a generally circular (when viewed from the top) ring with curved inner and outer walls. The inner and outer walls can be spherical. In some embodiments, an upper portion of the snap ring 130 has generally cylindrical inner and/or outer walls and a lower portion of the snap ring 130 has generally spherical inner and/or outer walls. As shown in FIGS. 2A and 2B, the outer wall of the snap ring 130 is generally sized and shaped to correspond to an inner wall of the lower portion 124 of the housing 120, and the inner wall of the snap ring 130 is generally sized and shaped to correspond to the head 112 of the screw 110. The snap ring 130 has a slit 132. The slit 132 allows the snap ring 130 to flex and expand to be placed around the enlarged head 112 of the screw 110 during assembly.

As shown in FIGS. 1-2B, the saddle 140 can have a generally cylindrical outer surface. An upper surface of the saddle 140 has an indentation 142 sized and shaped to receive the rod in use. The indentation 142 can be shaped approximately as a portion of a cylinder. As shown, the indentation 142 can cause an upper portion of the saddle 140 to be generally U-shaped with two opposing projections or arms. A lower surface of the saddle 140 has an indentation 144 sized and shaped to receive the enlarged head 112 of the screw 110. An outer surface of the saddle 140 can include one or more indentations 148. In the illustrated embodiment, the outer surface of the saddle 140 includes two indentations 148, one in each of the arms of the upper portion of the saddle 140 such that the indentations 148 are positioned opposite each other. Each of the indentations 148 can receive a pin during assembly as described in greater detail herein. The saddle 140 also includes a through hole 146 that allows a screwdriver to reach the proximal end of the head 112 in use. Although in the illustrated embodiment the saddle 140 is a unitary piece, in other embodiments, the saddle 140 can have two or more separate parts.

The screw 110, housing 120, snap ring 130, and saddle 140 can be preassembled. The screw 110 can be loaded into the lower opening 128. The snap ring 130 can be loaded into the upper opening 126 and pushed over and onto the head 112 of the screw 110. The screw 110 and snap ring 130 assembly is then pulled or pushed down in the housing 120, for example, such that the snap ring 130 is positioned adjacent the lower opening 128 as shown in FIGS. 1-2B. The saddle 140 can then be loaded into the housing 120 such that the indentation 142 is aligned with the third and fourth openings 160, 162 of the housing 120 and the indentations 148 are aligned with the holes 125 in the housing 120. Pins 141 are press-fit into the holes 125 and indentations 148 to secure the screw 110, housing 120, snap ring 130, and saddle 140 together. When assembled, the screw 112 and snap ring 130 can rotate and pivot polyaxially with respect to the housing 120. Alternatively, the screw 112 can rotate and pivot polyaxially with respect to the snap ring 130, which may be able to rotate and pivot polyaxially with respect to the housing 120 or may be fixed relative to the housing 120. In some embodiments, the pins 141 provide a downward force on the saddle 140, which then presses downward on the head 112 of the screw 110. This creates friction between the snap ring 130 and the housing 120. The screw 110 therefore generally does not rotate or pivot relative to the housing 120 unless the friction force is overcome, for example, by the surgeon or other user physically moving the screw 110 or housing 120 relative to the other.

In use, two or more screw assemblies 100 can be secured to two or more adjacent vertebrae, for example, in the pedicles of adjacent vertebrae, by threading the shaft 114 into the bone. A rod can then be placed in the third and fourth openings 160, 162 of the housings 120 and on the saddles 140 to link the two or more screw assemblies 100. In some embodiments, the rod can be approximately straight. In other embodiments, the rod can be curved. The rod can be of various lengths and diameters. For example, the length can be selected based on the number of adjacent vertebrae the rod is intended to span. Once the rod is in place, set screws can be threaded into the upper portions 122 of the housings 120 to secure the rod and lock the housings 120 and rod in place in a chosen orientation.

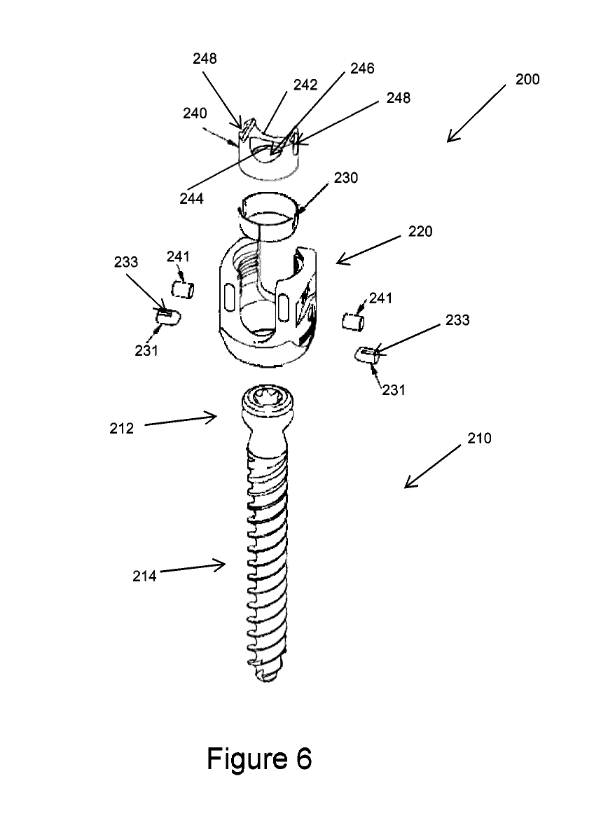

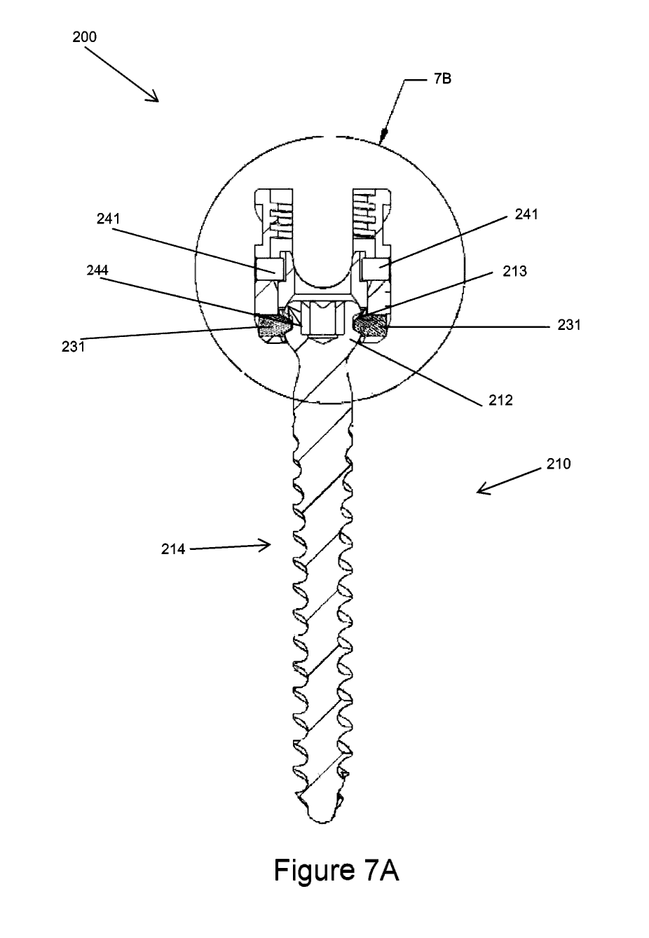

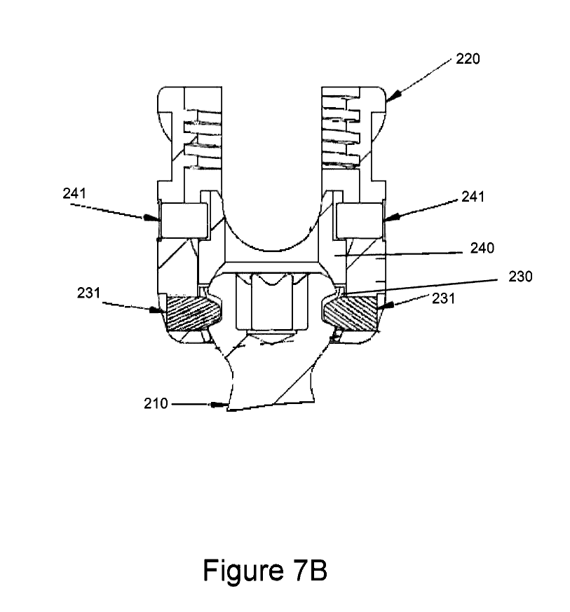

FIGS. 6-7B illustrate an alternative embodiment of a screw assembly 200. Similar to screw assembly 100, screw assembly 200 includes a screw 210 configured to be secured to a vertebra, a housing 220, a snap ring 230, and a saddle 240. The screw assembly 200 can further include a set screw and a spinal rod. In some embodiments, one or more of the components described herein is made of a metal, such as titanium or alloys thereof For example, one or more components can be made at least partially of titanium 6AL 4V ELI. In some embodiments, the housing 220 is made of cobalt-chrome. In some embodiments, the spinal rod can be made of cobalt-chrome and/or titanium or a titanium alloy.

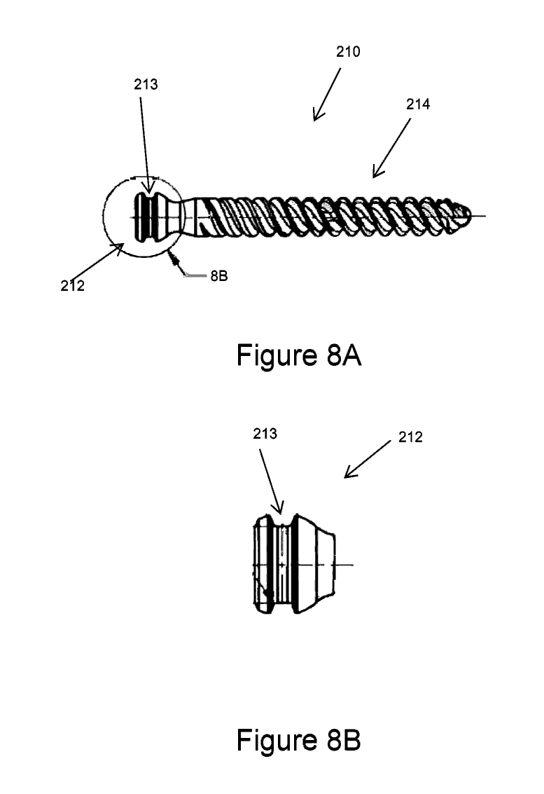

The screw 210 has an enlarged head 212 at a proximal end and a shaft or body portion 214 extending from the head 212 to a tip at a distal end. As shown in FIGS. 8A-8B, the head 112 has a groove 213 extending around the circumference of the head 112. The head 112 can also have a flattened proximal end or surface that can receive a screwdriver. The shaft 214 can be at least partially threaded and adapted to be implanted into a patient's spine, for example, into the pedicle of a vertebra. In some embodiments, the shaft 114 has a triple lead thread configuration, for example as described above with respect to screw 110. At least a portion of the screw shaft 214 can be coated with a hydroxyapatite (HA) coating as described above with respect to screw 110.

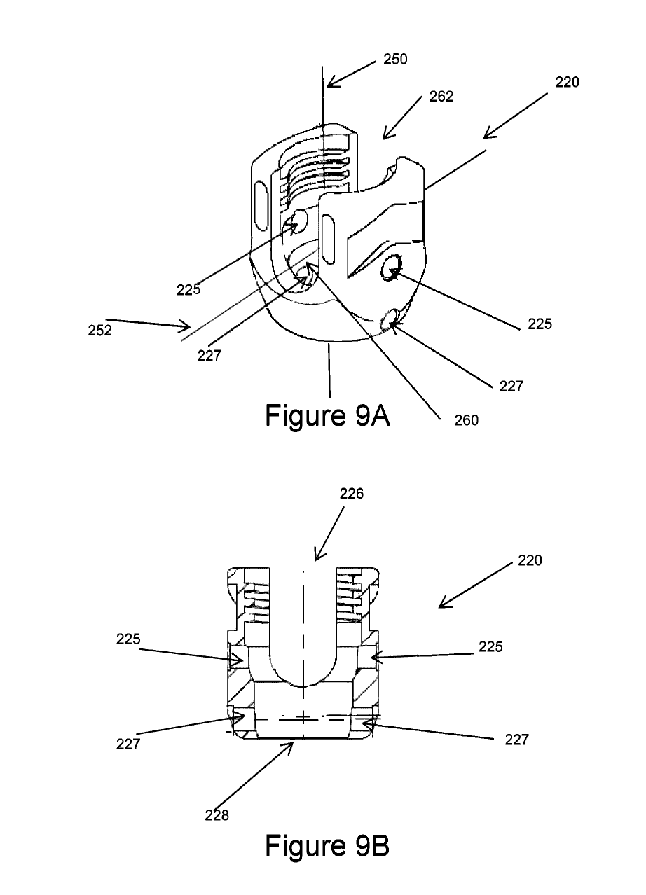

As shown in FIGS. 9A-9B, the housing 220 is similar to housing 120 and can include many or all of the features shown and described herein with respect to housing 120. In the illustrated embodiment, the lower portion of the housing 220 also includes one or more holes 227 extending perpendicularly to the first axis 250 and second axis 252. In the illustrated embodiment, the lower portion includes two holes 227 positioned opposite each other, each hole 227 generally vertically aligned with one of the holes 225.

As shown in FIGS. 6-7B, the saddle 240 is similar to saddle 140 and can include many or all of the features shown and described herein with respect to saddle 140.

For example, the saddle 240 has a generally cylindrical outer surface, an indentation 242 in the upper surface sized and shaped to receive the rod in use, an indentation 244 in the lower surface sized and shaped to receive the enlarged head 212 of the screw 210, a through hole 246, and two indentations 248 that each receives a pin 241 during assembly.

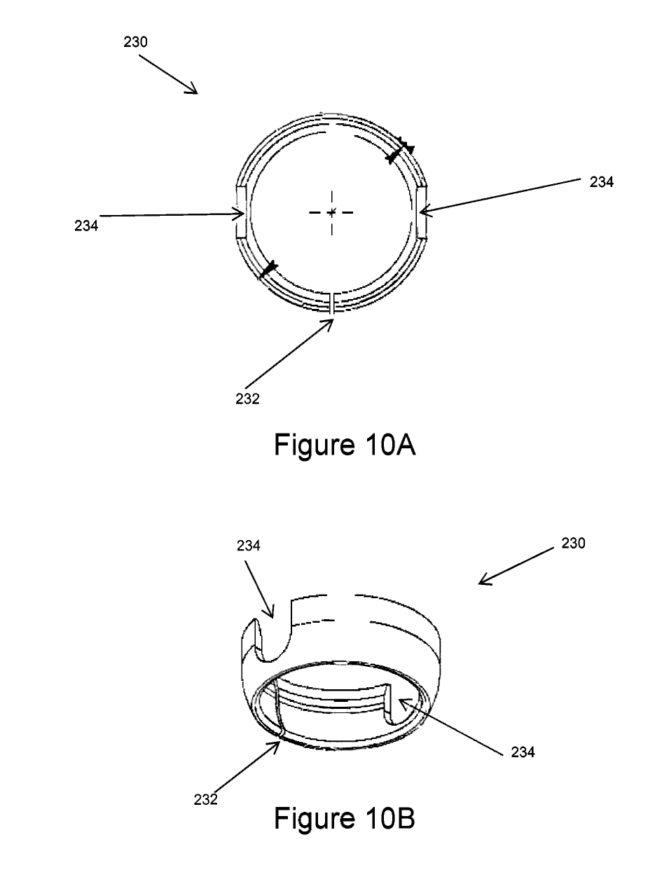

As shown in FIGS. 10A-10B, the snap ring 230 is a generally circular (when viewed from the top) ring with curved inner and outer walls. In some embodiments, an upper portion of the snap ring 230 has generally cylindrical inner and/or outer walls and a lower portion of the snap ring 230 has generally spherical inner and/or outer walls. As shown in FIGS. 7A and 7B, the outer wall of the snap ring 230 is generally sized and shaped to correspond to an inner wall of the lower portion of the housing 220, and the inner wall of the snap ring 230 is generally sized and shaped to correspond to the head 212 of the screw 210. The snap ring 230 has a slit 232. The slit 232 allows the snap ring 230 to flex and expand to be placed around the enlarged head 212 of the screw 210 during assembly. The snap ring 230 can also include one or more indentations or cutouts 234 extending from an upper surface of the snap ring 230. In the illustrated embodiment, the snap ring 230 includes two cutouts 234 positioned opposite each other. In the illustrated embodiment, the slit 232 is between the cutouts 234 (i.e., 90° clockwise from one of the cutouts 234 and 90° counterclockwise from the other of the cutouts 234), although other configurations and arrangements are also possible.

The screw 210, housing 120, snap ring 230, and saddle 240 can be preassembled. The screw 210 can be loaded into the lower opening 228. The snap ring 230 can be loaded into the upper opening 226 such that the cutouts 234 are aligned with the holes 227 and pushed over and onto the head 212 of the screw 210. The screw 210 and snap ring 230 assembly is then pulled or pushed down in the housing 220, for example, such that the snap ring 230 is positioned adjacent the lower opening 228 as shown in FIGS. 7-7B. The saddle 240 can then be loaded into the housing 220 such that the indentation 242 is aligned with the third and fourth openings 260, 262 of the housing 220 and the indentations 248 are aligned with the holes 225 in the housing 120. Pins 241 are press-fit into the holes 225 and indentations 248 to secure the screw 210, housing 220, snap ring 230, and saddle 240 together. Uniplanar pins 231 are press-fit into the holes 227, cutouts 234, and groove 213 in the head 212 of the screw 210. As shown in FIG. 6, the uniplanar pins 231 can have flat portions 233. The flat portions 233 engage corresponding flat portions within the holes 227 of the housing 220 to control rotational orientation of the uniplanar pins 231 relative to the housing 220 and screw 210. Once assembled, the screw 210 can rotate relative to the housing 220 about a longitudinal axis of the screw 210; however, interaction of the uniplanar pins 231 and the groove 213 limit pivoting of the screw 210 relative to the housing 220 to a single plane. The plane of movement of the screw 210 relative to the housing 220 can be perpendicular to the uniplanar pins 231 and parallel to the rod when disposed in the housing 220.

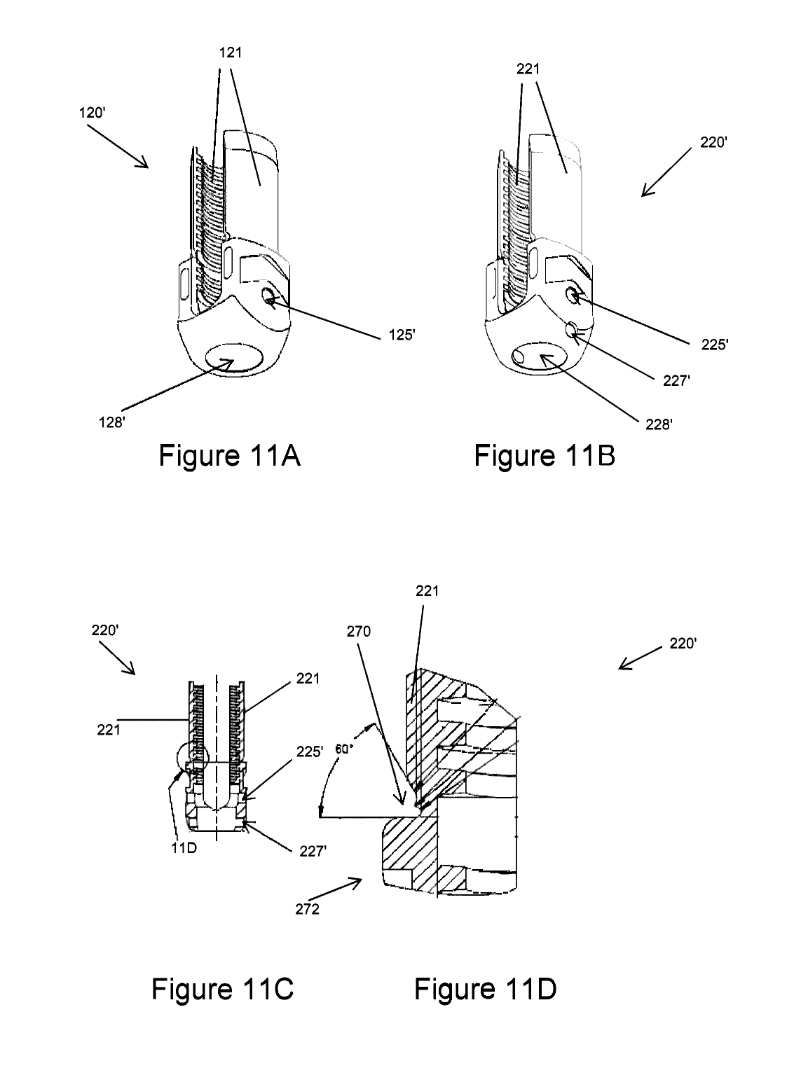

In some embodiments, a screw assembly according to the present disclosure can be a reduction screw. FIG. 11A illustrates an example embodiment of a housing 120′ for a reduction screw. The housing 120′ is similar to housing 120 and can include many or all of the features of housing 120. However, the housing 120′ also includes two extended tabs 121. One of the extended tabs 121 extends from each of the arms of the housing 120′. In the illustrated embodiment, the extended tabs 121 are curved and generally partially cylindrically shaped; however, other shapes or configurations are also possible. The extended tabs 121 can advantageously help guide the rod into the housing 120′. As shown, the extended tabs 121 can be internally threaded. The internally threaded extended tabs 121 can therefore receive the set screw and allow the set screw to be threaded down into the housing 120′ once the rod has been delivered. FIG. 11B illustrates another example embodiment of a housing 220′ for a reduction screw. The housing 220′ is similar to housing 220 and can include many or all of the features of housing 220. However, the housing 220′ also includes extended tabs 221 like the extended tabs 121 of the housing 120′.

FIG. 11C illustrates a cross-sectional view of housing 220′, and FIG. 11D illustrates a detail view of the area indicated by circle 11D in FIG. 11C. The housing 220′ includes a frangible or weakened section 270 between a base portion 272 of the housing 220′ and each of the extended tabs 221. In use, once the rod and set screw have been secured in the base portion 272, the extended tabs 221 can be broken off at the frangible sections 270.

In some embodiments, a screw assembly according to the present disclosure, including any of the screw assemblies described herein, can be modular. For example, one or more of the components (e.g., screw, housing, snap ring, and/or saddle) can be provided to the surgeon separately from one or more of the other components rather than preassembled. Such a modular screw can be at least partially or fully assembled within the patient. This can advantageously help increase the surgeon's visibility during the procedure, which can, for example, allow the surgeon to more thoroughly decorticate the surrounding fusion bed in the surrounding bone.

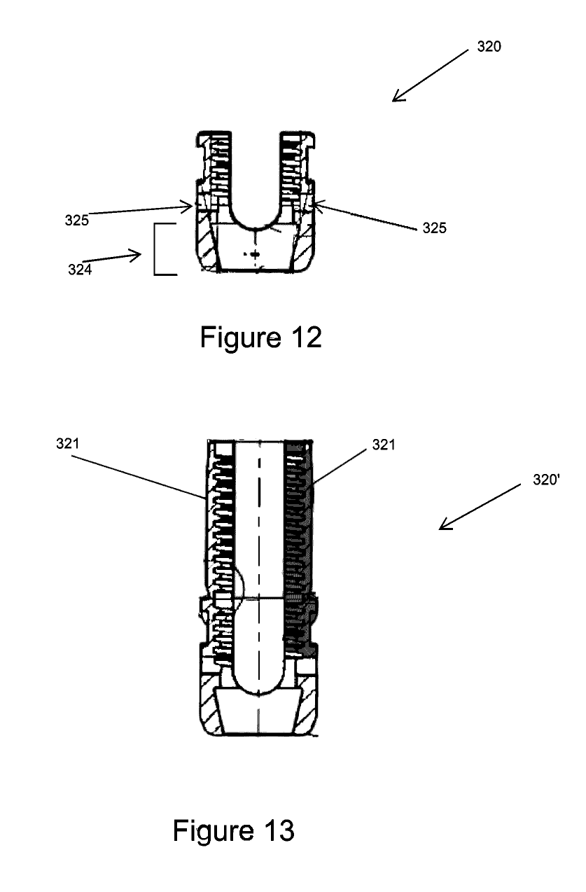

FIG. 12 illustrates an example embodiment of a housing 320 that can be used in a modular screw assembly. Housing 320 can be similar to housing 120 and can include many or all of the features of housing 120 (e.g., holes 325 that receive pins 141 that engage the saddle). In the illustrated embodiment, an interior surface of the lower portion 324 is tapered. FIG. 13 illustrates an example embodiment of a housing 320′ that can be used in a modular reduction screw assembly. Similar to housing 320, the lower portion of the housing 320′ has a tapered interior surface. As shown, the housing 320′ includes extended tabs 321.

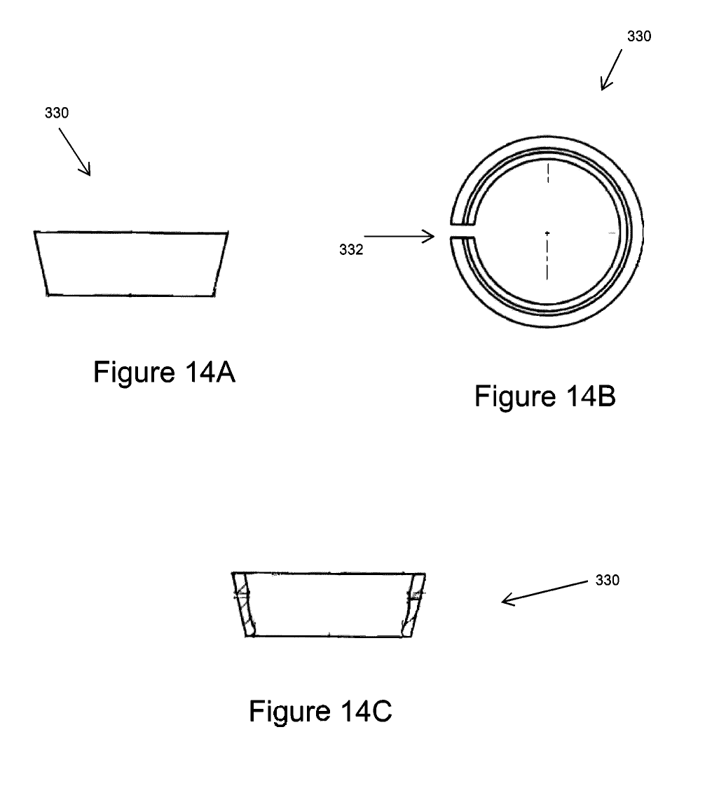

FIGS. 14A-14C illustrate an example embodiment of a snap ring 330 that can be used in a modular screw assembly (e.g., with housing 320 or housing 320′). The snap ring 330 includes a slit 332 that allows the snap ring 330 to flex and expand to be placed around the enlarged head of the screw. In the illustrated embodiment, an outer surface of the snap ring is 330 is tapered to correspond to the tapered interior surface of the lower portion 324 of the housing 320, 320′.

In some embodiments, a modular screw assembly can be provided with the screw and snap ring 330 preassembled, e.g., with the snap ring 330 pre-attached to or disposed around the enlarged head of the screw, e.g., via a friction fit. In some embodiments, the housing 320, 320′ can be provided preassembled with the saddle (e.g., the saddle can be pre-attached to the housing 320, 320′ with the pins 141). In use, the surgeon can select a screw (e.g., screw 110) and snap ring 330 sub-assembly (e.g., select a particular size or length screw) and secure the screw and snap ring 330 sub-assembly to a vertebra in the patient. The surgeon can then select a housing 320, 320′ or housing 320, 320′ and saddle sub-assembly and couple the housing 320, 320′ (or housing 320, 320′ and saddle sub-assembly) to the screw and snap ring 330 sub-assembly, which is implanted in the patient's body, for example, by sliding or pressing the tapered interior surface of the lower portion 324 of the housing 320, 320′ onto the tapered outer surface of the snap ring 330. The screw and snap ring 330 sub-assembly and housing 320, 320′ sub-assembly can be secured together via, e.g., a snap fit, friction fit, and/or by the rod and set screw once the rod and set screw are delivered and secured to the screw assembly. In some embodiments, a modular screw assembly can be provided with the housing 320, 320′, snap ring 330, and saddle preassembled (e.g., with the pins 141). In use, the surgeon can select a screw (e.g., screw 110) and secure the screw to a vertebra in the patient. The surgeon can select a housing 320, 320′ pre-assembled with the snap ring 330 and saddle and couple the housing 320, 320′ with the snap ring 330 and saddle to the screw, for example, by sliding or pressing the housing 320, 320′ and snap ring 330 onto the enlarged head of the screw. The screw and housing 320, 320′ with the snap ring 330 and saddle can be secured together via, e.g., a snap fit, friction fit, or other suitable means.

Similar to other embodiments described herein, the housing 320, 320′ can be made of cobalt-chrome. The screw in a modular screw assembly can have a proximal tapered and/or triple lead thread configuration as described herein. For example, screw 110 can be used in a modular screw assembly. In some embodiments, the screw in a modular screw assembly can be coated with a HA coating as described herein.

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this invention may comprise, additional to its essential features described herein, one or more features as described herein from each other embodiment of the invention disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise.

The scope of the present disclosure is not intended to be limited by the specific disclosures of preferred embodiments in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

What is claimed is:

1. A pedicle screw comprising:
a screw having a threaded shaft and an enlarged head at a proximal end, wherein the shaft is coated with a hydroxyapatite coating; and
a housing having an upper portion with an upper opening and a lower portion with a lower opening extending along a first axis of the housing, wherein the enlarged head of the screw is disposed within the housing and the shaft extends out of the housing through the lower opening, the housing having a third opening and a fourth opening along a second axis transverse to the first axis adapted to receive an elongated rod.

2. The pedicle screw of claim 1, wherein only a portion of the shaft is coated with a hydroxyapatite coating.

3. The pedicle screw of claim 1, wherein a distal portion of the shaft has a relatively thinner hydroxyapatite coating, and a proximal portion of the shaft has a relatively thicker hydroxyapatite coating.

4. The pedicle screw of claim 1, wherein each surface of each thread revolution of the shaft is uniformly coated with the hydroxyapatite coating.

5. The pedicle screw of claim 1, wherein a thickness of the hydroxyapatite coating on proximally-facing flanks is different than a thickness of the hydroxyapatite coating on distally-facing flanks.

6. The pedicle screw of claim 1, wherein only a crest and either a proximally-facing flank or a distally-facing flank of each thread revolution of the shaft is coated with the hydroxyapatite coating.

7. The pedicle screw of claim 1, wherein the threaded shaft has a triple lead thread configuration.

8. The pedicle screw of claim 1, further comprising a snap ring positioned around the enlarged head of the screw and in the lower portion of the housing adjacent the lower opening.

9. The pedicle screw of claim 1, further comprising a saddle disposed in the housing and having a generally cylindrical outer surface, an upper surface having a partially cylindrically shaped indentation configured to receive the elongated rod, and a lower surface having an indentation configured to receive the enlarged head.

10. The pedicle screw of claim 1, wherein the upper portion of the housing is internally threaded and configured to receive an externally threaded set screw.

11. The pedicle screw of claim 1, wherein the screw can rotate and pivot polyaxially relative to the housing prior to the rod being secured within the housing.

12. The pedicle screw of claim 1, wherein the screw can rotate and pivot uniaxially relative to the housing prior to the rod being secured within the housing.

13. The pedicle screw of claim 1, wherein the housing comprises two extended tabs extending from opposite sides of the housing, wherein the extended tabs are configured to guide the rod into the housing.

14. The pedicle screw of claim 13, wherein the extended tabs are internally threaded.

15. A method of manufacturing a bone screw assembly, the method comprising:
providing a bone screw and a housing, wherein the bone screw comprises an enlarged head and a shaft having a threaded portion, and wherein the housing is configured to receive the bone screw such that the enlarged head is disposed in a lower portion of the housing and the shaft extends from a lower opening of the housing; and
applying a hydroxyapatite coating to the threaded portion of the bone screw.

16. The method of claim 15, wherein applying the hydroxyapatite coating comprises spray coating the threaded portion of the bone screw with the hydroxyapatite coating.

17. A modular pedicle screw assembly comprising:
a screw having a threaded shaft and an enlarged head at a proximal end; and
a housing provided separately from the screw, the housing having an upper portion with an upper opening and a lower portion with a lower opening extending along a first axis of the housing, the housing having a third opening and a fourth opening along a second axis transverse to the first axis adapted to receive an elongated rod,
wherein the housing is configured to be secured to the screw when the screw is secured to a patient's vertebra such that the enlarged head of the screw is disposed within the housing and the shaft extends out of the housing through the lower opening.

18. The modular screw assembly of claim 17, further comprising a snap ring provided with the housing and positioned in the lower portion of the housing, wherein the snap ring is configured to be positioned around the enlarged head of the screw when the housing is secured to the screw.

19. The modular screw assembly of claim 18, wherein an interior surface of the lower portion of the housing is tapered.

20. The modular screw assembly of claim 19, wherein an outer surface of the snap ring is tapered.

21. A method of securing a pedicle screw to a patient's vertebra, the method comprising:
securing a screw to the vertebra, the screw having a threaded shaft and an enlarged head at a proximal end; and
securing a housing to the screw when the screw is secured to the vertebra, wherein the housing is provided separately from the screw, the housing having an upper portion with an upper opening and a lower portion with a lower opening extending along a first axis of the housing, the housing having a third opening and a fourth opening along a second axis transverse to the first axis adapted to receive an elongated rod, wherein when the housing is secured to the screw, the enlarged head of the screw is disposed within the housing and the shaft extends out of the housing through the lower opening.

22. The method of claim 21, wherein a snap ring is provided with the housing and positioned in the lower portion of the housing, and wherein the snap ring is configured to be positioned around the enlarged head of the screw when the housing is secured to the screw.
